(19) United States
(12) Patent Application Publication
(10) Pub. No.: US 2012/0328715 A1
(43) Pub. Date: Dec. 27, 2012

(54) PREVENTION AND TREATMENT OF OSTEOCHONDROSIS IN ANIMALS AND HUMANS

(76) Inventor: Edgar W. Johnson, Urbana, IL (US)

(21) Appl. No.: 13/587,677

(22) Filed: Aug. 16, 2012

Related U.S. Application Data
(62) Division of application No. 11/916,449, filed on May 21, 2008.
(60) Provisional application No. 60/687,653, filed on Jun. 2, 2005.

(51) Int. Cl. A61K 33/22; A61P 3/00; A61P 19/00; C01B 35/18; C01B 35/10

(52) U.S. Cl. 424/658; 423/279; 423/283; 424/659

(57) ABSTRACT

A method of preventing or treating osteochondrosis comprising administering a therapeutically effective amount of a boron containing compound to a mammal in need of such treatment.

### CROSS REFERENCE TO RELATED APPLICATION

This application is a division of application Ser. No. 11/916,449, filed May 21, 2008, which is a U.S. National Phasing patent application of International Application PCT/US/2006/021505, having the international filing date of Dec. 6, 2006, which claims the benefit under 35 USC 119(e) of U.S. Provisional Application No. 60/687,653, filed Jun. 2, 2005, all of which are incorporated in its entirety herein by reference.

### BACKGROUND OF THE INVENTION

Lameness is a major cause of culling and death in female pigs of breeding age, affecting over 20 million animals annually. At least 3 to 10% of young growing swine die or are culled due to lameness. Osteochondrosis (OC) is a major factor in this lameness, causing economic losses potentially exceeding $200 million in the United States alone.

OC is a non-infectious disease of cartilage affecting young growing animals and humans. OC is characterized by abnormal development of articular cartilages of the joints and in the growth plates of the bones, with associated changes in bone development. Lameness occurs when OC changes cause pain and/or interfere with normal skeletal function.

OC is the major cause of lameness in swine. It has been reported that 20 to 80 percent or more of growing pigs are affected by OC. OC severe enough to cause lameness is observed in 5 to 10 percent of horses and large breed dogs, and in 1 of 40 humans. OC is also reported in young growing cattle, especially bulls, and in sheep. OC is not common in cats but has been reported.

In humans, OC primarily afflicts adolescents, an age group that is very physically active and has bones which are still growing. The disease is more common among boys than girls. In children between the ages of 10 to 15, the disease frequently appears at the elbow, knee, or foot joints. Afflicted humans experience tenderness, swelling, and pain at the affected joints which worsens with activity.

Among the more common forms of OC in human children are: Freiberg's disease, which occurs in the head of the metatarsals of the feet in children between the ages of 12-15; Legg-Calve-Perthes' disease, which occurs in the hip in children between the ages of 6 to 9; Osgood-Schlatter disease, which occurs in the tibial tubercle apophysis at the insertion of the patellar tendon in the knee in children between the ages of 10 to 15; Panner's disease, which occurs in the capitellum of the distal humerus at the elbow in children between the ages of 510; and Sinding-Larsen-Johannson disease, which occurs at the inferior pole of the patella in the knee in children between the of ages 10-15. Animal correlates of each human OC condition are observed, with species and breed related “predilection sites”. In particular, different specific joints are more likely to be affected in a given species or breed; for instance there is a tendency for “elbow dysplasia” to develop in German Shepherds.

Thus, improved methods for the prevention and treatment of OC would be of great economic value in the livestock industry, would reduce animal suffering, and would help alleviate the painful joint discomfort and loss of function and mobility experienced by humans suffering from this disease.

Furthermore, phosphate pollution resulting from excess phosphorus in animal feed is an increasing problem. Such phosphorus can potentially contaminate ground water. There is a need to provide animal feed with reduced phosphorus content to reduce ground water contamination. Reduction in phosphorus use would assist animal producers in complying with nutrient control regulations.

It would be highly advantageous if one could provide a reduced phosphorus animal feed that would simultaneously facilitate the prevention and treatment of OC.

### BRIEF SUMMARY OF THE PREFERRED EMBODIMENTS OF THE INVENTION

The inventors have made the unexpected discovery that the administration of boron containing compounds is effective in preventing and treating osteochondrosis in animals. In one embodiment, this invention provides an animal feed containing supplemental boron. Animal feeds contain plant material. Boron is a required element for plant growth. As such all plants and hence all plant material in animal feeds contain some boron, e.g. 10-20 ppm boron in com/soybean feed (unless the boron as been extracted). The animal feeds of the present invention contain supplemental boron in addition to the boron naturally present in the animal feed from the plant material. The supplemental boron is supplied as a boron-containing compound, as plant material with elevated boron levels or as microorganisms such as yeast with elevated boron levels. Among the boron-containing compounds that may be used in the practice of the present invention are sodium borate and boric acid as typical boron sources. However, the invention is not limited to these forms of boron. Also included are other inorganic forms of boron such as calcium borate, as well as, organic boron compounds and complexes that dissociate or are metabolized in the body to release boron as borate or boric acid. Among the inorganic forms are sodium borate, boric acid, calcium borate, magnesium borate, halogen containing borate, ammonium borate, potassium borate, iron and magnesium containing borate, tantalum borate, beryllium borate, iron and nickel containing borate, carbonate containing borate, sodium and calcium containing borate, arsenate containing borate, calcium and rare earth containing borate, sulphate containing borate, magnesium and calcium containing borate, manganese borate, aluminum borate, calcium and strontium containing borate, phosphate containing borate, tin borate, strontium borate, zinc borate, calcium borosilicate, sodium borosilicate, aluminum borosilicate, calcium and rare earth containing borosilicate, lead borosilicate, barium borosilicate, lithium borosilicate, and sodium fluoroborate. Among the organic forms are complexes and compounds formed by boron, usually as boric acid, with fructose, sorbitol, mannitol, xylitol, sorbose, threonine, methionine, modified starches, hydrolyzed starches, oxidized starches, non-modified starches, dextrins, amidated sugars, glucosamine, mannosamine, esters of glycerol fatty acids, salicylate complexes, salts of bisoxalato acid, calcium borosucrose, alcohols, alcohol amines, sugar acids, saccharic acid, gluconic acid, aminated sugar acids, and calcium borogluconate. In this embodiment, the supplemental boron containing compounds are typically included in animal feed at concentrations providing about 1 to about 500 ppm supplemental elemental boron. In other embodiments, the boron containing compounds are typically included in animal feed at concentrations providing about 1 to about 150 ppm supplemental elemental boron. In yet another embodiment, the supplemental boron containing compounds are typically included in animal feed at concentrations providing about 50 ppm or about 25 to 50 ppm supplemental elemental boron. Among the animals that would benefit from the animal feed are pigs, horses, mules, donkeys, cattle, sheep, goats, llamas, dogs, and cats.

In a further unexpected discovery, the inventors have determined that the addition of supplemental boron to animal feed allows for the reduction in phosphorus content of the animal feed. Thus, in another embodiment, the invention provides an improved animal feed containing supplemental boron-containing compounds and reduced phosphorus content. In such an embodiment, the supplemental boron containing compound can be sodium borate or boric acid. However, the invention is not limited to these forms of supplemental boron. Other inorganic forms of boron such as calcium borate, as well as, organic boron compounds and complexes that dissociate or are metabolized in the body to release boron as borate or boric acid can be used as well. Among the inorganic forms are sodium borate, boric acid, calcium borate, magnesium borate, halogen containing borate, ammonium borate, potassium borate, iron and magnesium containing borate, tantalum borate, beryllium borate, iron and nickel containing borate, carbonate containing borate, sodium and calcium containing borate, arsenate containing borate, calcium and rare earth containing borate, sulphate containing borate, magnesium and calcium containing borate, manganese borate, aluminum borate, calcium and strontium containing borate, phosphate containing borate, tin borate, strontium borate, zinc borate,. calcium borosilicate, sodium borosilicate, aluminum borosilicate, calcium and rare earth containing borosilicate, lead borosilicate, barium borosilicate, lithium borosilicate, and sodium fluoroborate. Among these organic forms are complexes and compounds formed by boron, usually as boric acid, with fructose, sorbitol, mannitol, xylitol, sorbose, threonine, methionine, modified starches, hydrolyzed starches, oxidized starches, non-modified starches, dextrins, amidated sugars, glucosamine, mannosamine, esters of glycerol fatty acids, salicylate complexes, salts of bisoxalato acid, calcium borosucrose, alcohols, alcohol amines, sugar acids, saccharic acid, gluconic acid, aminated sugar acids, and calcium borogluconate. The boron can be combined with talc in a ratio of boron containing compound to talc of approximately 5:1, 6:1, 7:1, 8:1, 9:1, 10:1, 11:1, 12:1, 13:1, 14:1. 15:1, 16:1, 17:1, 18:1, 19:1, 20:1; 21:1; 22:1, 23:1; 24:1 or 25:1 prior to addition to the animal feed. The supplemental boron containing compounds are included in the animal feed at about 1 to about 500, about 1 to about 150 or about 50 ppm or about 25 to 50 ppm supplemental boron and the total phosphorus content is reduced by at least 3% as compared to a comparable animal feed without supplemental boron. Generally, the animal feed is supplemented with boron at concentrations ranging from about 5 to about 150 ppm. The animal feed is suitable for pigs, horses, mules, donkeys, cattle, sheep, goats, llamas, dogs, and cats among other animals.

In another embodiment, the invention provides a method of decreasing the amount of phosphorus excreted by an animal. In this embodiment, animals are fed a diet of an improved animal feed composition containing about 1 to about 500, about 1 to about 150 or about 50 ppm or about 25 to 50 ppm supplemental boron supplied as boron containing compounds, plant material with elevated boron levels, yeast or other microorganisms with elevated boron levels in which the animal feed composition has at least a 3% reduction in phosphorus as compared to a comparable animal feed without supplemental boron. Generally, the animal feed contains supplemental boron at concentrations ranging from 5-150 ppm. In such an embodiment, the supplemental boron containing compound can be sodium borate or boric acid can be used. However, other inorganic forms of boron such as calcium borate, as well as, organic boron compounds and complexes that dissociate or are metabolized in the body to release boron as borate or boric acid can be used. Among the inorganic forms are sodium borate, boric acid, calcium borate, magnesium borate, halogen borate, ammonium borate, potassium borate, iron and magnesium containing borate, tantalum borate, beryllium borate, iron and nickel containing borate, carbonate containing borate, sodium and calcium containing borate, arsenate containing borate, calcium and rare earth containing borate, sulphate containing borate, magnesium and calcium containing borate, manganese borate, aluminum borate, calcium and strontium containing borate, phosphate containing borate, tin borate, strontium borate, zinc borate, calcium borosilicate, sodium borosilicate, aluminum borosilicate, calcium and rare earth containing borosilicate, lead borosilicate, barium borosilicate, lithium borosilicate, and sodium fluoroborate. Among the organic forms are complexes and compounds formed by boron, usually as boric acid, with fructose, sorbitol, mannitol, xylitol, sorbose, threonine, methionine, modified starches, hydrolyzed starches, oxidized starches, non-modified starches, dextrins, amidated sugars, glucosamine, matmosamine, esters of glycerol fatty acids, salicylate complexes, salts of bisoxalato acid, calcium borosucrose, alcohols, alcohol amines, sugar acids, saccharic acid, gluconic acid, aminated sugar acids, and calcium borogluconate. The method is suitable for use with pigs, horses, mules, donkeys, cattle, sheep, goats, llamas, dogs, and cats among other animals.

An additional embodiment provides a method of increasing the efficiency of absorption of phosphorus in animals. In this embodiment, animals are fed a diet of an improved animal feed composition containing about 1 to about 500, about 1 to about 150 or about 50 ppm or about 25 to 50 ppm supplemental boron wherein the phosphorus absorption is improved by at least a 3% as compared to a comparable animal feed without supplemental boron. In such an embodiment, the supplemental boron containing compound can be sodium borate or boric acid. However, other inorganic forms of boron such as calcium borate, as well as, organic boron compounds and complexes that dissociate or are metabolized in the body to release boron as borate or boric acid can be used. Among the inorganic forms are sodium borate, boric acid, calcium borate, magnesium borate, halogen containing borate, ammonium borate, potassium borate, iron and magnesium containing borate, tantalum borate, beryllium borate, iron and nickel containing borate, carbonate containing borate, sodium and calcium containing borate, arsenate containing borate, calcium and rare earth containing borate, sulphate containing borate, magnesium and calcium containing borate, manganese borate, aluminum borate, calcium and strontium containing borate, phosphate containing borate, tin borate, strontium borate, zinc borate, calcium borosilicate, sodium borosilicate, aluminum borosilicate, calcium and rare earth containing borosilicate, lead borosilicate, barium borosilicate, lithium borosilicate, and sodium fluoroborate. Among the organic forms are complexes and compounds formed by boron, usually as boric acid, with fructose, sorbitol, mannitol, xylitol, sorbose, threonine, methionine, modified starches, hydrolyzed starches, oxidized starches, non-modified starches, dextrins, amidated sugars, glucosamine, mannosamine, esters of glycerol fatty acids, salicylate complexes, salts of bisoxalato acid, calcium borosucrose, alcohols, alcohol amines, sugar acids, saccharic acid, gluconic acid, aminated sugar acids, and calcium borogluconate. The method is suitable for use with pigs, horses, mules, donkeys, cattle, sheep, goats, llamas, dogs, and cats among other animals.

In yet an additional embodiment, this invention provides a method of reducing environmental phosphorus pollution from an animal farm. In this embodiment, animals are fed a diet of an improved animal feed composition containing 1 to about 500, about 1 to about 150 or about 50 ppm or about 25 to 50 ppm supplemental boron containing compounds whereby the phosphorus efflux is reduced by at least 3% as compared to that of a comparable animal feed without supplemental boron. In such an embodiment, the supplemental boron containing compound can be sodium borate or boric acid. However, inorganic forms of boron such as calcium borate, as well as, organic boron compounds and complexes that dissociate or are metabolized in the body to release boron as borate or boric acid can be used. Among the inorganic forms are sodium borate, boric acid, calcium borate, magnesium borate, halogen containing borate, ammonium borate, potassium borate, iron and magnesium containing borate, tantalum borate, beryllium borate, iron and nickel containing borate, carbonate containing borate, sodium and calcium containing borate, arsenate containing borate, calcium and rare earth containing borate, sulphate containing borate, magnesium and calcium containing borate, manganese borate, aluminum borate, calcium and strontium containing borate, phosphate containing borate, tin borate, strontium borate, zinc borate, calcium borosilicate, sodium borosilicate, aluminum borosilicate, calcium and rare earth containing borosilicate, lead borosilicate, barium borosilicate, lithium borosilicate, and sodium fluoroborate. Among the organic forms are complexes and compounds formed by boron, usually as boric acid, with fructose, sorbitol, mannitol, xylitol, sorbose, threonine, methionine, modified starches, hydrolyzed starches, oxidized starches, non-modified starches, dextrins, amidated sugars, glucosamine, mannosamine, esters of glycerol fatty acids, salicylate complexes, salts of bisoxalato acid, calcium borosucrose, alcohols, alcohol amines, sugar acids, saccharic acid, gluconic acid, aminated sugar acids, and calcium borogluconate. The method is suitable for use with pigs, horses, mules, donkeys, cattle, sheep, goats, llamas, dogs, and cats among other animals.

In a further embodiment, the invention also provides a method of treating or preventing OC by administering a therapeutically effective amount of a boron containing compound to a mammal in need of such treatment. In such an embodiment, the boron containing compound can be sodium borate or boric acid. However, the invention can be used with other inorganic forms of boron such as calcium borate, as well as, organic boron compounds and complexes that dissociate or are metabolized in the body to release boron as borate or boric acid can be used. Among the inorganic forms are sodium borate, boric acid, calcium borate, magnesium borate, halogen containing borate, ammonium borate, potassium borate, iron and magnesium containing borate, tantalum borate, beryllium borate, iron and nickel containing borate, carbonate containing borate, sodium and calcium containing borate, arsenate containing borate, calcium and rare earth containing borate, sulphate containing borate, magnesium and calcium containing borate, manganese borate, aluminum borate, calcium and strontium containing borate, phosphate containing borate, tin borate, strontium borate, zinc borate, calcium borosilicate, sodium borosilicate, aluminum borosilicate, calcium and rare earth containing borosilicate, lead borosilicate, barium borosilicate, lithium borosilicate, and sodium fluoroborate. Among the organic forms are complexes and compounds formed by boron, usually as boric acid, with fructose, sorbitol, mannitol, xylitol, sorbose, threonine, methionine, modified starches, hydrolyzed starches, oxidized starches, non-modified starches, dextrins, amidated sugars, glucosamine, mannosamine, esters of glycerol fatty acids, salicylate complexes, salts of bisoxalato acid, calcium borosucrose, alcohols, alcohol amines, sugar acids, saccharic acid, gluconic acid, aminated sugar acids, and calcium borogluconate. The mammal to be treated can be a human or an animal. The supplemental boron-containing compounds can be administered prior to the appearance of symptoms of osteochondrosis as a preventive measure. Among the animals that can benefit from this invention are pigs, horses, mules, donkeys, cattle, sheep, goats, llamas, dogs, and cats.

In another embodiment, the invention provides a method of decreasing the amount of pre-weaning mortality in animals. In another embodiment, the invention provides a method of improving reproductive rates of animals by increasing the rate of return to estrus and conception rates. In these embodiments, previously pregnant, pregnant, nursing and/or lactating animals are fed a diet of increased boron. The diet may contain about 1 to about 500, about 1 to about 150 or about 50 ppm or about 25 to 50 ppm supplemental boron containing compounds. The boron may be provided in improved animal feed composition or in milk or water. Generally, the milk, water or animal feed contains supplemental boron at concentrations ranging from 5-150 ppm. In such embodiments, the supplemental boron containing compound can be sodium borate or boric acid can be used. However, other inorganic forms of boron such as calcium borate, as well as, organic boron compounds and complexes that dissociate or are metabolized in the body to release boron as borate or boric acid can be used. Among the inorganic forms are sodium borate, boric acid, calcium borate, magnesium borate, halogen borate, ammonium borate, potassium borate, iron and magnesium containing borate, tantalum borate, beryllium borate, iron and nickel containing borate, carbonate containing borate, sodium and calcium containing borate, arsenate containing borate, calcium and rare earth containing borate, sulphate containing borate, magnesium and calcium containing borate, manganese borate, aluminum borate, calcium and strontium containing borate, phosphate containing borate, tin borate, strontium borate, zinc borate, calcium borosilicate, sodium borosilicate, aluminum borosilicate, calcium and rare earth containing borosilicate, lead borosilicate, barium borosilicate, lithium borosilicate, and sodium fluoroborate. Among the organic forms are complexes and compound formed by boron, usually as boric acid, with fructose, sorbitol, mannitol, xylitol, sorbose, threonine, methionine, modified starches, hydrolyzed starches, oxidized starches, non-modified starches, dextrins, amidated sugars, glucosamine, mannosamine, esters of glycerol fatty acids, salicylate complexes, salts of bisoxalato acid, calcium borosucrose, alcohols, alcohol amines, sugar acids, saccharic acid, gluconic acid, aminated sugar acids, and calcium borogluconate. The method is suitable for use with pigs, horses, mules, donkeys, cattle, sheep, goats, llamas, dogs, and cats among other animals.

In an additional embodiment, the boron-containing compounds are added to drinking water, mineral or vitamin supplements, in a milk formulation, or other food products for the treatment and prevention of OC and/or reduction in pre-weaning mortality.

In an additional embodiment, this invention provides a boron-talc composition where the ratio of boron-containing compound to talc is approximately 5:1, 6:1, 7:1, 8:1, 9:1, 10:1, 11:1, 12:1, 13:1, 14:1, 15:1, 16:1, 17:1, 18:1, 19:1, 20:1; 21:1, 22:1, 23:1, 24:1 or 25:1. In such an embodiment, the boron is a boron containing compound which can be sodium borate or boric acid. However, the invention is not limited to these forms of supplemental boron. Other inorganic forms of boron such as calcium borate, as well as, organic boron compounds and complexes that dissociate or are metabolized in the body to release boron as borate or boric acid can be used as well. Among the inorganic forms are sodium borate, boric acid, calcium borate, magnesium borate, halogen containing borate, ammonium borate, potassium borate, iron and magnesium containing borate, tantalum borate, beryllium borate, iron and nickel containing borate, carbonate containing borate, sodium and calcium containing borate, arsenate containing borate, calcium and rare earth containing borate, sulphate containing borate, magnesium and calcium containing borate, manganese borate, aluminum borate, calcium and strontium containing borate, phosphate containing borate, tin borate, strontium borate, zinc borate,. calcium borosilicate, sodium borosilicate, aluminum borosilicate, calcium and rare earth containing borosilicate, lead borosilicate, barium borosilicate, lithium borosilicate, and sodium fluoroborate. Among these organic forms are complexes and compounds formed by boron, usually as boric acid, with fructose, sorbitol, mannitol, xylitol, sorbose, threonine, methionine, modified starches, hydrolyzed starches, oxidized starches, non-modified starches, dextrins, amidated sugars, glucosamine, mannosamine, esters of glycerol fatty acids, salicylate complexes, salts of bisoxalato acid, calcium borosucrose, alcohols, alcohol amines, sugar acids, saccharic acid, gluconic acid, aminated sugar acids, and calcium borogluconate.

Talc is available for use in the present exemplary embodiments from a variety of commercial sources. For example, Luzenac America is a supplier of talc. Examples of talc products from Luzenac America include: E-Z Flow 40, E-Z-Flow MB, E-Z Flow MT, E-Z Flow RM, and E-Z Flow VT.

### BRIEF DESCRIPTION OF THE DRAWINGS

FIG. 1 is a graph indicating a reduction in the occurrence of osteochondrosis with supplemental boron treatment.

FIG. 2 is a graph showing the association between increasing osteochondrosis scores in the pig right hock with increasing soundness scores among pigs not receiving supplemental boron.

FIG. 3 is a graph showing the effect of supplemental boron treatment in the reduction of soundness scores associated with early growth.

FIGS. 4 and 5 are graphs which show that administration of 3-NPB along with boron resulted in a prevalence and severity of gross joint pathology similar to that observed in unsupplemented pigs.

### DETAILED DESCRIPTION OF THE INVENTION

### Introduction

Boron has long been known to be an essential plant nutrient, but a role for boron in human physiology has only recently been appreciated. The present inventors have discovered a beneficial effect of boron supplementation of animal and human diets. In particular, although previous studies had shown that boron containing compounds could alleviate the bone disease, osteoporosis, the present inventors have discovered that boron containing compounds also alleviate a disease of the joints and growth plate cartilages, osteochondrosis (OC).

Osteoporosis is a disease in which bones become fragile and become increasingly likely to break as the disease progresses. Osteoporosis, or porous bone, is characterized by low bone mass and structural deterioration of bone tissue, which leads to bone fragility and an increased susceptibility to fractures of the hip, spine, and wrist. Thus, osteoporosis is a disease that specifically strikes the bone, generally after full and normal development. Also, because of its progressive nature, osteoporosis is a disease that most commonly manifests itself in older individuals. One out of every two women and one in four men over the age of 50 will have an osteoporosis-related fracture in their lifetimes.

In contrast, osteochondrosis is a generalized skeletal disease of growing animals and results from a disturbance in the articular and growth plate cartilages. The bone is only secondarily affected. As a consequence, dyschondroplasia is technically a more correct term to describe this condition. A further condition, osteochondrosis dissecans, results in the chipping, fracturing and/or fragmentation of the articular surface. Osteochondrosis dissecans is thought to be caused by an underlying weakness in the cartilage caused by an osteochondrotic lesion. Lesions are characterized by focal impaired endochondondral ossification, resulting in areas of retained cartilage extending into the subchondral bone. See R. John Wardale and Victor C. Duance. Journal of Cell Science 107, 47-59 (1994).

Because of the differences in the pathobiology of OC and osteoporosis—the two diseases affect different aspects of the bone system and different age groups—one would not expect that a treatment that alleviates osteoporosis, a degenerative disease of the bones of the elderly, would help in the treatment of OC, a disease of the cartilage in joints of the young. The inventors have found that surprisingly, boron containing compounds are useful agents in the prevention and treatment of OC.

### Role of Boron In the Behavior of Cartilage

The extracellular matrix (ECM) of the articular cartilage provides cushioning between opposing bone surfaces at a joint in a mammalian limb. Synovial fluid is the fluid contained in joints. Synovial membranes line the joints, bursae, and tendon sheaths. The function of the synovial fluid is to lubricate the joint space and transport nutrients to the articular cartilage. The articular cartilage provides a low friction point of contact for the smooth flexing operation of the joints and also a cushioning function at joints, by absorbing the impact of shocks transmitted through the bones and supporting the weight of the animal. The cartilage is composed of a variety of components including proteoglycan and a collagen network in an aqueous environment.

Proteoglycans play a role in maintaining the cushioning seen at joints. The cartilage ECM is illustrated as a network of the collagen fibers which interlocks with and is interlocked by proteoglycan. The proteoglycan is a flexible gel-like material and the collagen forms a mesh-network that holds the proteoglycan in place. The proteoglycan provides compressive strength while tensile strength is provided by the collagen network. Proteoglycan in articular and growth plate cartilage contains large amounts of sulfated glycosaminoglycans (GAG) that have a strong negative charge. At physiologic pH, these negatively charged GAG molecules draw sodium ions and water into the ECM of the cartilage, causing the proteoglycan to “inflate”. The inflated proteoglycan provides buoyant pressure to resist compression, thus protecting the collagen network and underlying structures from compression damage. Better cushioning and thus greater compression resistance is provided by a fully hydrated proteoglycan complement and a fully extended and taut collagen network.

Without limiting this invention to any particular mechanism nor being bound by theory, one potential model for how boron functions in OC is the “standing hypothesis”. In this hypothesis, boron functions by crosslinking the proteoglycan into the extracellular matrix. One postulated mechanism for how this occurs is that boron provides for three-dimensional boroester crosslinking of carbohydrate, proteoglycan, glycoprotein, glycolipid, lipid, protein, and amino acid structures. In the case of extracellular membrane structures such as cartilage and neural tissue, this would include proteoglycans such as aggrecan (the large aggregating proteoglycan of cartilage), complex proteins such as collagen in its various forms and types, and associated proteins such as cartilage link protein. The crosslinking of the proteoglycan stabilizes and unifies the matrix, allowing for better distribution of compressive forces and prevention of proteoglycan loss, which would decrease the cushioning ability of the synovial membrane. In contrast, boron functions to prevent osteoporosis by increasing the plasma levels of hydroxylated steroids. See U.S. Pat. No. 4,849,220. Thus, one would not have predicted that boron would have an effect in treating a disease of the cartilage, such as OC, which has a totally different etiology from osteoporosis. In osteoporosis, the bone itself is directly affected. In OC, the cartilage is affected.

### Etiology And Pathology of OC

Although the precise cause of OC is not yet known, a number of mechanisms for the progression of this disease have been suggested. The influence of compressive forces in producing damage to the growing and transitioning cartilage appears to be a major factor. Studies in pigs have suggested that focal changes in blood supply during normal epiphyseal growth is central to the pathogenesis of osteochondrosis. Cartilage canals are temporary blood vessel-containing structures within growing cartilage. The canals gradually regress with age during the process of chondrification, wherein the blood vessels contained within the canals are replaced with cartilage. Formation of the lesions associated with osteochondrosis has been associated with the premature chondrification and regression of these canals. In particular, the premature disruption of the blood supply results in necrosis of the cartilage canal distal to the point of interruption. See Ytrehus et al. Bone 35: 1294-1306 (2004). Thus, it is not surprising that severe clinical osteochondrosis appears most commonly in fast-growing animals with rapid weight gain. See Wardale and Duance Journal of Cell Science 107: 47-59 (1994).

It has also been demonstrated in humans and dogs that in osteochondrosis, the proteoglycan of cartilage is resorbed by the action of matrix metalloproteinase-3 (M1VIP-3) derived from synovial membrane cells and chondrocytes. See Shinmei et al. 1991; Okada et al. 1992; Mehraban et al. 1994. Loss of proteoglycan from the extracellular matrix of the cartilage would lead to a decreased capacity of the cartilage to absorb and cushion compressive forces.

### Role of Boron In OC

In order to further probe the contribution of boron deficiency in OC, and to test whether boron functions in OC through quadrivalent crosslinking, 3-nitrophenylboronic acid (3-NPB) was administered to animals. 3-NPB blocks crosslinks by binding to sites normally occupied by boric acid or borate. The results are described in Example 4. 3-NPB treated animals had increased lameness and clinical manifestations of OC. The increase in lameness could be prevented by supplementing the diet with boron. These experiments show that OC is directly correlated to boron levels in pigs, horses, cattle, and dogs.

### Boron Compounds For the Treatment of OC In Animals

Given the widespread occurrence of OC in livestock and in particular, pigs, this invention discloses a safe and effective means of preventing and treating OC by providing for animal feed to be supplemented with boron containing compounds. It will be appreciated by one of skill in the art that animal feeds, derived at least in part from plant materials, will contain basal levels of boron since boron is a required element for plant growth. For instance, typical alfalfa contains about 37 ppm boron. Thus, the term supplemental boron as used herein refers to exogenously added boron that supplements the basal levels of boron already present in commonly used animal feeds. When the term boron is used in this disclosure, it can denote both elemental boron and boron containing compounds. The boron containing compounds useful for the practice of this invention may include any suitable organic or inorganic boron containing compounds, including boron containing minerals. Among the preferred forms of boron are sodium borate and boric acid. Other useful inorganic forms of boron include calcium borate. One of skill in the art will recognize that other inorganic forms of boron that may be used in this invention include borates with: magnesium, halogen, ammonium, potassium, iron and magnesium, tantalum, beryllium and nickel, carbonate, sodium and calcium, arsenate, calcium and rare earth, sulphate, magnesium and calcium, manganese, aluminum, calcium and strontium, phosphate, tin, zinc, and strontium. Other forms include: borosilicates or silicoborates with calcium, sodium, aluminum, calcium and rare earth, lead, barium, lithium, and fluoroborate with sodium. Natural inorganic boron containing compounds are known to skilled artisans by various mineral names such as borax, colemanite, hydroboracite, kernite, ulexite, datolite, danburite, szaibelyite, suanite, inderite, sassolite, inyoite, probertite, howlite, ezcurrite, kurnakovite, meyerhofferite, priceite, nobleite, and searlesite to name but a few such designations. A listing of inorganic borate compounds and minerals can be found in Supplement to Mellor's Comprehensive Treatise on Inorganic and Theoretical Chemistry, Volume V Boron, by Joseph William Mellor, Longman Group Limited, London, 1980.

Examples of organic boron-containing compounds are well known to those of skill in the art. Examples of such organic boron-containing compounds are found in U.S. Pat. Nos. 4,312,989, 4,499,082, and 5,312,816 all of which are hereby incorporated by reference. Among the forms of organic boron that would be useful in the practice of this invention are organic boron complexes such as boron threonine, boron methionine, and boron ascorbate, as well as boron complexed with other amino acids. These amino acids can include the 20 common amino acids that are specified by the genetic code, as well as variant and modified amino acids which are not encoded by the genetic code. These are examples of organic forms of boron that are rapidly metabolized to release borate or boric acid. Other useful forms of organic boron are boron carbohydrate complexes such as those disclosed in U.S. Pat. No. 5,962,049. Among the carbohydrates that form useful complexes with boron include saccharides such as fructose, sorbitol, mannitol, xylitol, and sorbose. A commercially available form of boron complexed with fructose is Fruitex arm available from FutureCeuticals and described in U.S. Pat. No. 5,962,049.

Other organic forms of boron that can be used in the practice of this invention include: borated modified starches (such as hydrolyzed or oxidized starches), borate non-modified starches, borated dextrins, borated amidated sugars (such as glucosamine or mannosamine), borate esters of glycerol fatty acids, borate-salicylate complexes, salts of bisoxalato borate (such as sodium or potassium salts), calcium borosucrose, borate esters (such as (RO) 3B), alcohol amine borate esters, and borate complexes with sugar acids (such as saccharic acid and gluconic acid), and borate complexes with aminated sugar acids. One particularly desirable sugar acid to use in this invention is calcium borogluconate. Yet another form of boron are anion exchange resins which can be boronated. One such resin which can be boronated is Amberlite™.

It will be appreciated by one of skill in the art that when a particular boron containing compound is described herein, it is intended that all possible solvates, pharmaceutically acceptable salts, esters, amides, complexes, chelates, stereoisomers, geometric isomers, crystalline or amorphous forms, metabolites, metabolic precursors or prodrugs of the compound are also separately described by a chemical structural formula or chemical name. Furthermore, if any of the boron containing compounds described herein contain stereochemistry, all enantiomeric and diastereomeric forms of the compound are intended. Thus, when applicable, boron containing compounds may occur as racemates, racemic mixtures and as individual diastereomers, or enantiomers with all isomeric forms being included. A racemate or racemic mixture does not necessarily imply a 50:50 mixture of stereoisomers.

Furthermore, it will be appreciated by one of skill in the art that the borates of the present invention will encompass many different grades, including those that are FDA and non-FDA approved. Thus, among the grades of borates that can be used in the practice of this invention are: pharmaceutical or formulary grade, nuclear grade, fertilizer grade, industrial grade, pesticidal grade, and special quality (SQ) grade.

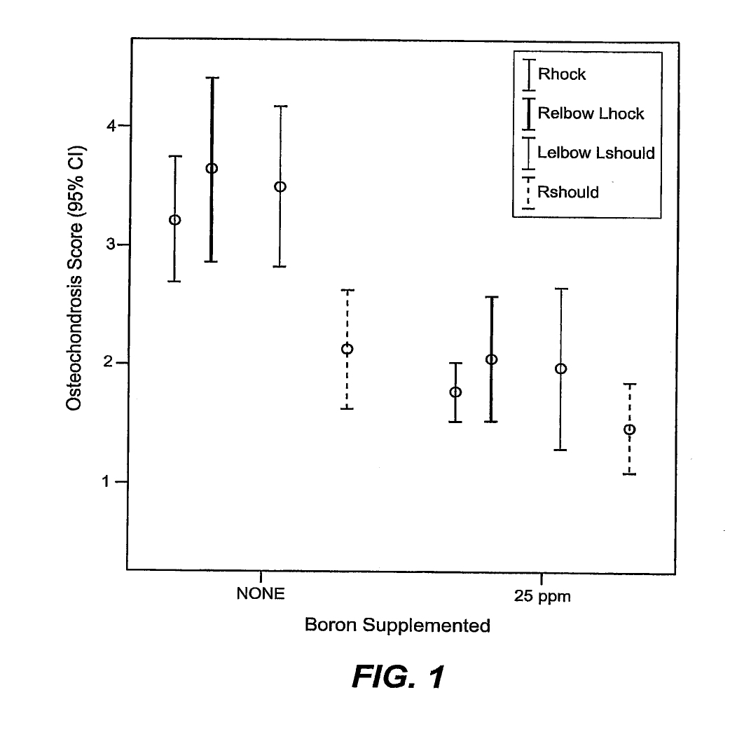

Suitable ranges for use of the boron containing compounds includes the supplementation of boron in animal feed from about 1 to about 500 ppm above that naturally present in the animal feed. Another suitable range for supplementation is about 1 to about 150 ppm. As shown in FIGS. 1, 3 and 4, the inventors have found that supplemental boron at 25 ppm to 50 ppm provides a significant reduction in the occurrence of OC in pigs. Accordingly, in one embodiment, this invention provides an animal feed composition that is supplemented with 25 ppm to 50 ppm boron containing compounds. In one embodiment, the supplemental boron containing compound is sodium borate. In another embodiment, the supplemental boron containing compound is boric acid. It will be clear to one of skill in the art that other concentrations of boron may be used depending on the severity of the disease or animal to be treated. Furthermore, it will be clear to one of skill in the art that other supplemental boron containing compounds may also be used in the practice of this invention.

The boron described herein may be combined with talc. The boron-containing compound to talc ratio may be approximately 5:1, 6:1, 7:1, 8:1, 9:1, 10:1, 11:1, 12:1, 13:1, 14:1, 15:1, 16:1, 17:1, 18:1, 19:1, 20:1; 21:1,22:1, 23:1, 24:1 or 25:1.

### Incorporation of Supplemental Boron Into Animal Feeds

A variety of methods are known in the art for the production of animal feeds. These various methods can be adapted to allow inclusion of supplemental boron into the feed in amounts that will have a beneficial effect on OC when fed to animals.

For example, supplemental boron in the amounts disclosed above can be incorporated into animal feed compositions such as those described in U.S. Pat. No. 3,946,109. Alternatively, a variety of other feed compositions are commercially available from suppliers such as Purina, ADM, Land O'Lakes, and Moorman's. Supplemental boron can be mixed into a composition of choice using for instance, the mixing methods disclosed in U.S. Pat. No. 4,189,240. The composition containing supplemental boron can be used to form animal feed food blocks such as those disclosed in U.S. Pat. No. 5,120,565. Alternatively, supplemental boron can be incorporated into an animal feed composition which is formed by methods such as spray drying as disclosed in U.S. Pat. No. 4,777,240. The citation of these patents is solely to illustrate various methods available in the art for incorporating supplemental boron into an animal feed product and is not meant to limit the practice of the invention to the use of any one or more of these methods.

Other boron sources that can be incorporated into animal feeds to practice this invention include yeast preparations that are high in boron. It is already common practice to incorporate yeast into animal feeds. Hence, it would be fairly straightforward to include yeast with elevated boron levels in animal feed. Alternatively, crops that have been grown in soils with elevated boron levels can be harvested specifically for the purpose of serving as an enhanced boron source that can be incorporated into animal feeds. Such elevated boron levels may be naturally in the soil, may result from boron pollution, or may be added to the soil by fertilization or other means. Alternatively, supplemental boron containing compounds can be added to supplements, base mixes, and premixes that also contain vitamins and minerals. Such supplements, mixes, or premixes are typically added at an amount to constitute 0.5% to 30% of the final animal feed composition. In such an embodiment, the elemental boron concentration would be much higher (from about 3 times to 200 times higher) prior to dilution in the animal feed to result in a supplemental boron equivalent of 1500 ppm over a total daily ration.

Another alternative is to supplement animal feeds with foods, such as alfalfa, grapes, or coffee grounds, which are naturally high in boron content. Additionally, these and other foods can be manipulated to contain higher levels of boron by growth under elevated boron conditions as described above or by means of transgenic plant technology or other recombinant methods.

In a further embodiment, supplemental boron containing compounds can be provided as dietary supplements that can be directly hand-fed or “top-dressed” onto an animal feed. Such an embodiment could be in a formulation that contains other nutrients, excipients, or flavors. As an example, an equine nutrient supplement containing supplemental boron and other vitamins and minerals could be fed to a horse with a small spoon or cup or in the form of a bar or pellet. Alternatively, the supplement could be placed on top of or mixed in the animal's feed.

Such boron can be supplied to animal feeds as a boron-talc composition. The ratio of boron-containing compound to talc in the boron-talc composition can be approximately 5:1, 6:1, 7:1, 8:1, 9:1, 10:1, 11:1, 12:1, 13:1, 14:1, 15:1, 16:1, 17:1, 18:1, 19:1, 20:1; 21:1, 22:1, 23:1, 24:1 or 25:1

Should it be necessary, boron levels can be precisely determined by a variety of methods known in the art. U.S. Patent Publication 20040020840 and the patents disclosed therein describe a number of such methods.

### Reduction of Phosphorus Content of Animal Feeds And Improvement of Pre-Weaning Mortality

Phosphate pollution resulting from excess phosphorus in animal feed is an increasing problem. For example, approximately 70% of the phosphorus in a typical corn/soybean meal diet is unavailable to pigs, according to the National Research Council's 1998 Nutrient Requirements for Swine. This unavailable phosphorus ends up being excreted in manure. The high phosphate content of swine manure contributes to the environmental pollution associated with pig farming. Reducing the amount of excreted nutrients, particularly phosphorus, in swine production systems is an environmental priority and an important economic issue facing the swine industry. Thus, a means to increase the bioavailability of phosphorus in feed ingredients used to formulate swine rations would be desirable. The inventors have found that inclusion of supplemental boron in pig feed results in an increased absorption and utilization of the phosphorus present in pig diets. Supplemental boron promotes the efficient incorporation of phosphate into the calcium phosphate (hydroxyapatite) of bones. This effect is expected to be true in other animals as well.

By increasing the efficiency of absorption and utilization of phosphorus from animal diets such as pig diets, the inventors have found that the amount of phosphorus in typical pig feed formulations can be reduced. These results are shown in Example 3. The increased utilization of phosphorus from pig diets coupled with the reduction in the starting amount of phosphorus in pig feed can be expected to contribute to a reduction of phosphate pollution that results from pig farming. Thus, the inclusion of supplemental boron in animal feed as taught by this invention will not only contribute to the prevention and treatment of OC, it will also contribute to pollution reduction. While the foregoing discussion has focused on pigs, this invention is not limited to the reduction of phosphorus from pig feed exclusively. Rather, one of skill in the art will recognize that reduction of phosphorus use is applicable to all animals.

The formation of most bones of the axial skeleton begins with the formation of a cartilage model which is calcified and remodeled into bone that is mineralized with calcium and phosphate. Supplemental boron enhances the efficiency of this process. The postulated mechanism by which supplemental boron enhances the efficiency of the process is through stabilization of the extracellular matrix, although there may be other mechanisms.

Supplemental boron improves the efficiency of cartilage transformation/bone mineralization which improves the structural integrity of bone and bone mineralization characteristics. Calcium is added to diets at a level that promotes sufficient bone strength. The level of calcium that promotes optimum bone strength also paradoxically inhibits the intestinal absorption of phosphorus. Phosphorus absorption is also more efficient when dietary phosphorus level is reduced. The inventors have discovered that addition of supplemental boron to animal feed promotes bone mineralization and permits a proportional 3 to 5% reduction of both calcium and phosphorus in the animal feed while maintaining bone strength.

The 1998 NRC report on “Nutrient Requirements of Swine”, data from which is shown below in Table 1, indicates that typical requirements for calcium and phosphorus will vary during the lifetime of a pig. At earlier stages, when the bones of the skeleton are still undergoing development, greater amounts of calcium and phosphorus are needed to support increased bone growth. The requirements for calcium and phosphorus decrease as a pig matures and bone development is completed. Although the data presented below are for pigs, similar trends in requirements for calcium and phosphorus are observed during the life cycle of other animals.

TABLE 1

| Growing Pigs (NRC, 1998) | | | | | | |
|---|---|---|---|---|---|---|
| Body Weight (kg) | 3-5 | 5-10 | 10-20 | 20-50 | 50-80 | 80-120 |
| Calcium (%) | 0.90 | 0.80 | 0.70 | 0.60 | 0.50 | 0.45 |
| Phosphorus, total (%) | 0.70 | 0.65 | 0.60 | 0.50 | 0.45 | 0.4.0 |

| | |
|---|---|
| Sows | |
| Calcium (%) | 0.75 |
| Phosphorus, total (%) | 0.60 |

The inclusion of supplemental boron in the diet of various animals allows the levels of calcium and phosphorus to be reduced by at least 3% throughout the life cycle of animals. Thus, while the ratio of calcium to phosphorus is generally kept constant at each weight range indicated in Table 1, the absolute amounts of calcium and phosphorus can be lowered by at least 3% due to the addition of supplemental boron containing compounds.

Thus, in another embodiment, this invention provides an animal feed containing supplemental boron with a reduced level of phosphorus. In one embodiment, the supplemental boron is preferably provided at a concentration of about 1 to about 500 ppm elemental boron and the phosphorus level is reduced by 3 to 5% as compared to comparable animal feed without supplemental boron. The calcium level is generally reduced comparably to the phosphorus level. However, it will be recognized that if the supplemental boron containing compound is supplied as a calcium salt, such as calcium borate or calcium borogluconate, levels of calcium in the animal feed can also be correspondingly reduced. One such compound, calcium borogluconate, is already in use to treat hypocalcemia in cattle, sheep, and goats. In another embodiment, the supplemental boron concentration is preferably about 1 about 150 ppm and the phosphorus level is reduced by 3 to 5% as compared to comparable animal feed without supplemental boron. In yet another embodiment, the supplemental boron concentration is preferably about 25 ppm to 50 ppm and the phosphorus level is reduced by 3 to 5% as compared to comparable animal feed without supplemental boron.

In another embodiment, the invention provides a method of decreasing the amount of pre-weaning mortality by animals. In this embodiment, pregnant, nursing or lactating animals are fed a diet of an improved animal feed composition containing about 1 to about 500, about 1 to about 150 or about 50 ppm or about 25 to 50 ppm supplemental boron containing compounds in which the animal feed composition has at least a 3% reduction in phosphorus as compared to a comparable animal feed without supplemental boron. Generally, the animal feed contains supplemental boron at concentrations ranging from 5-150 ppm. In such an embodiment, the supplemental boron containing compound can be sodium borate or boric acid can be used or other inorganic forms of boron as described herein.

### OC In Humans

Osteochondrosis with its various manifestations has been found to be strikingly similar in six species of animals in which it has been reported. This has prompted experts to assert that it would be expected that osteochondrosis in humans would have the same etiology, pathogenesis, and pathology as has been observed in animals. See Olsson, S. E. and Reiland, S. 1978. The nature of osteochondrosis in animals—summary and conclusions with comparative aspects on osteochondrosis dissecans in man. Acta Radiologica Supplement No. 358:299-306.

Osteochondrosis in humans is defined in Dorland's Medical Dictionary as follows: a disease of the growth or ossification centers in children that begins as a degeneration or necrosis followed by regeneration or recalcification. Also called epiphyseal ischemic necrosis (q.v.), it may affect (1) the calcaneus (os calcis), a condition sometimes called apophysitis; (2) the capitular epiphysis (head) of the femur, a condition known as Legg-Calve-Perthes disease, Perthes disease, Waldenstrom's disease, coxa plana, and pseudocoxalgia; (3) the ilium; (4) the lunate (semilunar) bone, known as Kienbock's disease; (5) head of the second metatarsal bone, known as Freiberg's infraction; (6) the navicular (tarsal scaphoid); (7) the tuberosity of the tibia, called Osgood-Schlatter disease and Schlafter's disease; (8) the vertebrae, called Scheuermann's disease or kyphosis, juvenile kyphosis, vertebral epiphysitis, and kyphosis dorsalis juvenilis; (9) the capitellum of the humerus, called Panner's disease.

The locations of the joints affected in human children can be contrasted with regions affected in the pig. In the pig, osteochondrosis can be located in the following areas, listed in descending order of severity of lesions: 1. Articular-epiphyseal lesions: stifle, elbow, lumbar synovial intervertebral joints, hock, shoulder, and hip, 2. Growth plate lesions: distal ulna, distal femur, costochondral junction, femoral head, humeral head, ischiatic tuberosity, and thoracolumbar vertebrae, 3. Epiphysiolysis and apophysiolysis lesions: glenoid cavity, ischiatic tuberosity, capital femoral epiphysis, vertebral epiphyses, anconeal process, and distal ulnar epiphysis.

Because of the similarity of the disease in pigs and humans, another embodiment of the invention is the treatment and prevention of OC in humans. Boron compounds can be administered to patients suffering from OC. The boron containing compounds useful for the practice of this invention may include any suitable organic, inorganic, or mineral boron containing compounds. Among the preferred forms of boron are sodium borate and boric acid. Other useful inorganic forms of boron include calcium borate. Examples of organic boron-containing compounds are well known to those of skill in the art. Examples of such organic boron-containing compounds are found in U.S. Pat. Nos. 4,312,989, 4,499,082, and 5,312,816. Dosages that may find use in humans include 1-13 ppm.

### Formulations of Boron For Use In Humans

Described below are administration methods that are useful for humans. One particularly useful administration method is the provision of boron as mineral or vitamin supplements, for example, in food or pill format. However, it will be appreciated that many of the methods disclosed below, while especially applicable to humans, can also be used for the administration of boron to animals as well.

One especially useful form of administration for the boron containing compounds of the present invention is as a mineral supplement with vitamins that can be taken orally as a pill or added to food. Multi-vitamin and mineral supplements are useful in the maintenance and improvement of health by insuring adequate intake of micronutrients that are needed for disease prevention and to compensate for nutritional deficiencies that result from factors as inadequate dietary intake of essential nutrients. Vitamin and mineral preparations are commonly administered as general nutritional supplements or to treat specific medical conditions. Accordingly, the supplemental boron containing compounds of the present invention can be administered as mineral supplements with vitamins such as vitamin A, vitamin C, vitamin D, vitamin E, vitamin K, vitamin B1, vitamin B2, niacinamide, vitamin B6, vitamin B12, biotin, pantothenic acid, carnitine, silicon, molybdenum, germanium iron, phosphorus, iodine, magnesium, zinc, selenium, copper, chromium, potassium, choline, lycopene, and co-enzyme Q-10. Examples of mineral supplement formulations to which supplemental boron containing compounds can be added can be found in U.S. Pat. Nos. 4,752,479, 5,869,084, and 6,361,800. Such supplements containing the boron compounds of the present invention can be administered as chewable vitamin pills, or as supplements that can be added to beverages, or as supplements that can be added to foods.

In practicing the method of the present invention, the boron compounds may be administered per se or as components of a pharmaceutically acceptable composition. When used in medicine, the form of the supplemental boron compounds should be both pharmacologically and pharmaceutically acceptable.

Thus, the present invention may be practiced with the boron compounds being provided in pharmaceutical formulations, both for veterinary and for human medical use, comprising the active agent (the boron compound) together with one or more pharmaceutically acceptable carriers thereof and optionally any other therapeutic ingredients. The carrier(s) must be pharmaceutically acceptable in the sense of being compatible with the other ingredients of the formulation and not unsuitably deleterious to the recipient thereof. The active agent is provided in an amount effective to achieve the desired pharmacological effect, as described above, and in a quantity appropriate to achieve the desired daily dose.

The formulations include those suitable for oral, rectal, topical, nasal, ophthalmic, or parenteral (including subcutaneous, intramuscular, and intravenous) administration. Formulations suitable for parenteral administration are preferred.

The formulations may conveniently be presented in unit dosage form and may be prepared by any of the methods well known in the art of pharmacy. All methods include the step of bringing the active compound into association with a carrier which constitutes one or more accessory ingredients. In general, the formulations may be prepared by uniformly and intimately bringing the active compounds into association with a liquid carrier, a finely divided solid carrier, or both, and then, if necessary, shaping the product into desired formulations.

Formulations of the present invention suitable for oral administration may be presented as discrete units such as capsules, cachets, tablets, or lozenges, each containing a predetermined amount of the active ingredient as a powder or in the form of granules; or as a suspension in an aqueous liquor or a non-aqueous liquid, such as a syrup, an elixir, an emulsion, or a draught.

A tablet may be made by compression or molding, optionally with one or more accessory ingredients. Compressed tablets may be prepared by compressing in a suitable machine, with the active compound being in a free-flowing form such as a powder or granules which optionally is mixed with a binder, disintegrant, lubricant, inert diluent, surface active agent, or discharging agent. Molded tablets comprised of a mixture of the powdered active compound with a suitable carrier may be made by molding in a suitable machine.

One desirable formulation of the composition for administration is in a powdered form for dissolution or dilution with water or another suitable beverage or liquid before use. Alternatively, the composition can be contained in a ready to use form as part of a fortified beverage in liquid form. Also, boron containing compounds can be added to milk replacers. The composition can also be contained in a pudding with a custard or flan like texture or in the form of a bar suitable for ready consumption.

A syrup may be made by adding the active compound to a concentrated aqueous solution of a sugar, for example sucrose, to which may also be added any accessory ingredient(s). Such accessory ingredient(s) may include flavorings, suitable preservatives, agents to retard crystallization of the sugar, and agents to increase the solubility of any other ingredient, such as a polyhydroxy alcohol, for example glycerol or sorbitol.

Formulations suitable for parenteral administration conveniently comprise a sterile aqueous preparation of the active compound, which preferably is isotonic with the blood of the recipient (e.g., physiological saline solution).

Nasal spray formulations comprise purified aqueous solutions of the active compound with preservative agents and isotonic agents. Such formulations preferably are adjusted to a pH and isotonic state compatible with the nasal mucous membranes.

Formulations for rectal administration may be presented as a suppository with a suitable carrier such as cocoa butter, hydrogenated fats, or hydrogenated fatty carboxylic acids.

Topical formulations comprise the active compound dissolved or suspended in one or more media, such as mineral oil, petroleum, polyhydroxy alcohols, or other bases used for topical pharmaceutical formulations.

In addition to the aforementioned ingredients, the formulations of this invention may further include one or more accessory ingredient(s) selected from diluents, buffers, flavoring agents, binders, disintegrants, surface active agents, thickeners, lubricants, preservatives (including antioxidants), and the like.

The following examples further demonstrate several preferred embodiments of this invention. While the examples illustrate the invention, they are not intended to limit the invention. The patents cited herein are incorporated by reference in their entirety.

### EXAMPLE 1

### Boron Supplementation And Its Effects On OC-Associated Lameness In Swine

### Materials And Methods

Three groups of 19 pigs, Duroc and Yorkshire pigs were randomly blocked by breed, litter and weight. The basal diet consisted of commercial corn-soy diet containing 10 ppm boron.

Test diet group B was fed a basal diet plus 25 mg/kg boron as sodium borate decahydrate (borax). Test diet group A was fed a basal diet plus 25 mg/kg boron as sodium borate decahydrate (borax) and 250 mg/kg ascorbic acid.

Pigs were weighed at the beginning of the study, 4 weeks later and every 3 weeks until the end of the study. Animals were scored for soundness on a 5-point scale at each weighing. (Five-point scale: 1=no soundness defects; 2=minor soundness issues but still sound enough for retention as breeding animal; 3=not sound enough for retention for breeding but still marketable; 4=unsound, likely to be rejected at slaughter; 5=severely lame, requiring euthanasia for humane reasons.) Grading was done by the caretaker and the investigator at all weighing times. An experienced independent treatment-blind rater also evaluated the soundness of each animal and the ratings he provided were compared with the ratings of the caretaker and investigator by use of Cohen's Kappa test.

The animals were housed in a modern curtain sided barn with deep straw bedding, with ad libitum access to feed and water. Waterers and feeders were on a concrete pad but the remainder of the flooring was deep straw over a ground limestone and sand base. Floor space allowances exceeded the recommendations of the Guide for the Care and Use of Agricultural Animals in Agricultural Research and Teaching, First Revised Edition, 1999. Federation of Animal Science Societies. Savoy, Ill.

Pigs in the study were fed a typical corn-soybean meal diet which contained a proprietary commercial supplement from the Moorman's company at the manufacturer's suggested inclusion rate. The basal diet (no boron supplemented) was analysed and found to contain boron at the rate of 10 ppm which is typical for a corn-soybean meal based diet.

Animals were observed twice daily by the caretaker.

In mid October one pig was observed to be severely lame and was euthanized and necropsied. Three additional pigs, one lame Duroc, a sound York and a sound Duroc (littermate to the lame pig) were euthanized and necropsied for observational purposes in mid-November.

At the end of the study the pigs were transported to a laboratory where they were euthanized and necropsied. Six pigs in each of group B and group A were put on the control diet for 7 days at the end of the study.

At necropsy, samples of liver, heart, kidney, fat, skeletal muscle, proximal tibia, blood, and the rostral snout were retained and frozen at −40 ° C. prior to chemical analysis. All joints of the axial skeleton were evaluated for the presence of gross lesions of osteochondrosis and graded on a 5 point scale. (5-point scale: 1=no gross abnormalities; 2=minor imperfections in articular conformation or articular reddening present but no cartilage erosions; 3=cartilage intact but surface irregularities of the cartilage are present; 4—fissuring or erosion of articular cartilage is obvious; 5=full thickness cartilage lesions or cartilage flaps, osteochondritis dissecans obviously present).

The proximal femur (femoral head) and articular surfaces and growth plates associated with the stifle, hock, shoulder, elbow and carpus were sectioned with a band saw and approx. 0.5 to 1.0 cm sections fixed in formalin. Bone sections were decalcified with formic acid/sodium citrate, embedded in paraffin and sectioned at 5 microns. Sections were deparaffinized according to standard procedures. Two sections were made from each joint and growth plate and stained with either hematoxylin/eosin (H&E) or with toluidine blue (pH 4)/fast green for evaluation of the articular cartilage, subchondral bone and growth plate.

Mean joint lesion scores, growth rate and soundness scores by treatment and by factors boron and ascorbate were compared using an analysis of variance and t-test as appropriate. Soundness scores were dichotomised into binary categorical variables for lameness (soundness score >2) and for absence of defects (soundness <2), and the binary variables analysed by chi-square and logistic regression.

Tissue specimens stained with H&E were examined microscopically for the presence of lesions of osteochondrosis. Toluidine Blue staining allowed assessment of retention or loss of proteoglycan from the extracellular matrix (ECM) of the cartilage. Each tissue section was scored by a treatment-blind board-certified pathologist.

Further, specific joints and structures are selected for more detailed histomorphometric analysis. Both epiphyseal and growth plate specimens were obtained from boron treated and untreated animals.

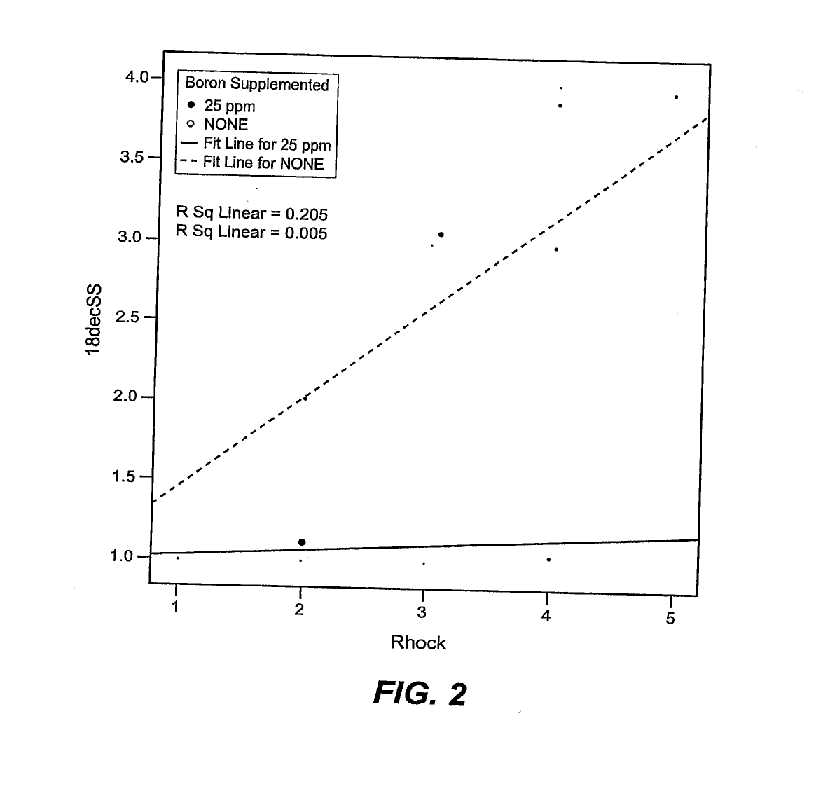

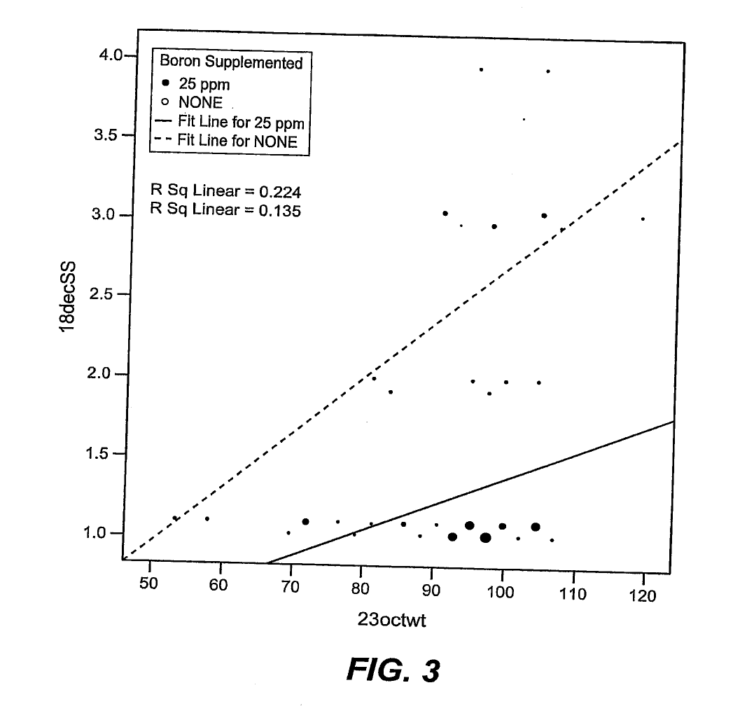

The results show that boron supplementation can be effective in reduction of the incidence of osteochondrosis-associated lameness in growing swine. Animals supplemented with boron had healthier joints than those receiving the basal diet with no supplemental boron (FIG. 1). Increasing soundness score (higher score=increasing lameness/leg unsoundness) is associated with increasing lameness in the pigs not receiving boron (FIG. 2). FIG. 3 illustrates the effect of early rapid growth (weight on 23 October) on soundness scores at the termination of the study (18 December). Pigs that did not receive boron and grew rapidly tended to develop leg unsoundness and lameness. Boron supplementation was useful in cartilage protection and prevention of lameness in rapidly growing swine, whereas the untreated group displayed a high prevalence of lameness and leg unsoundness that was clearly associated with the presence of cartilage damage typical of osteochondrosis in swine.

Anecdotal evidence from continuing experimental use of boron added to feed or drinking water indicates a consistent and sustained positive response in swine in a variety of production settings and genetics. This evidence is described in Example 5.

### EXAMPLE 2

### Reproductive Effects In Female Swine

It was observed that when sows were fed diets containing 50 ppm supplemental boron during the late gestation and early lactation period, piglet quality as assessed by uniformity, growth, and general thrift was improved, and pre-weaning piglet mortality was reduced. A preliminary pilot study confirmed these observations. Sows were fed a standard corn-soy diet. Half of the sows received an oral administration of a boron supplement to provide 1 mg boron per kg body weight. The other half did not receive any supplementation. Preliminary analysis of the data from the first 600 pigs indicated that the provision of boron to the gestating and lactating sows reduced pre-weaning mortality from 23% to 16% and increased piglet weight at 12 days of age from 8.0 pounds to 8.5 pounds, as compared to the non-supplemented groups.

To test the effect of boron on sows and their litters, a trial was established in a large commercial swine operation during an outbreak of porcine viral reproductive and respiratory disease (PRRSV). Boron was administered orally to a group of 51 sows individually housed in crates at the rate of 1 mg boron per pound BW per day beginning 1 week prior to farrowing and continuing until the piglets were weaned at 14 days of age. Their performance was compared to that of cohort of 50 sows of identical genetics and identical housing and husbandry conditions that did not receive boron. Boron treatment was supplied as a single daily dosing and was discontinued at weaning. All sows were fed a standard commercial sow diet.

There were no effects on litter size at birth or piglet birth weight. Piglets raised by sows consuming boron weighed 9.01 lbs at 12 days of age as compared with 8.32 lbs for the piglets raised by control sows (p=0.06). Piglet mortality in the boron treated group was 15.2% as compared with 20.3% among the controls (p=0.03). Sows that were fed boron returned to estrus an average of 1.6 days quicker than control sows (p=0.047). Boron treated sows were 1.2 times as likely as control sows to conceive (p=0.04). This result would be expected to have significant positive impact in a commercial pig raising operation.

It is postulated that borate exerts its beneficial effects in OCD prevention by modulation and stabilization of the extracellular matrix (ECM). In tissues like cartilage that possess an abundant ECM consisting of proteoglycan and collagen, the main effect of boron is likely mediated by a change in the mechanic (material) properties of the cartilage ECM. However many other tissues with important functions do also possess ECM components and extracellular receptors, the structure of which may be stabilized by boron cross-links which improves their functionality in cell to cell signaling, receptor functions, and adhesion functions. It is postulated that the effect of borate on reproduction is modulated by this sort of mechanism.

### EXAMPLE 3

### Effects of Boron On Phosphorus Digestibility And Excretion And Feed Conversion

A 28-day feeding trial was conducted in a large commercial farm setting with 144 crossbred pigs of initial body weight of 24 kg. Pigs were randomly allocated to 24 pens of 6 pigs per pen in a thermoneutral controlled environment barn with steel grid flooring. Each pen was equipped with a single-hole feeder. Water was available free-choice from a nipple drinker. Pigs were fed a commercial pig diet containing 0.5% phosphorus plus 0 or 50 mg/kg Boron and a calcium level of either 0.5 or 0.65% in a 2×2 factorial design. Feces were collected on the last 3 days of the study from each pen and a pooled aliquot was dried and submitted for chemical analysis. Yttrium oxide was added to the diet at 0.05% and served as a marker for phosphorus digestibility. Pig growth and feed consumption was measured at the end of the study with the pen as the experimental unit. Data was analyzed for effects of boron and calcium by univariate and multivariate analysis of variance and t-tests.

Supplemental boron increased average daily gain, improved feed conversion ratio and phosphorus digestibility, and reduced fecal phosphorus excretion per unit of growth (p<0.05). (Table 2). Daily feed intake was not significantly modified by boron or calcium level (p>0.20). Decreasing calcium level improved feed conversion (p<0.05). There were no significant interactive effects of boron x calcium on feed conversion or phosphorus excretion (p>0.25).

The digestibility and fecal excretion of phosphorus is of significant concern to animal agriculture. Phosphorus is a costly dietary ingredient and the phosphorus in animal wastes is a potential environmental pollutant. In the present study, the phosphorus excretion per unit of production was reduced by 15% by adding boron to the diet. Boron also produced a significant effect on overall feed conversion ratio. It is expected that these effects of boron would have a significant impact in reducing environmental pollution as well as reducing production costs in animal agriculture

TABLE 2
Effects of Boron Supplementation

| | No Added B | 50 ppm Added B | S.E. | p |
|---|---|---|---|---|
| Feed | 2.65 | 2.32 | 0.082 | 0.033 |
| Phosphorus | 33.4 | 36.3 | 0.820 | 0.017 |
| Phosphorus | 88.1 | 74.6 | 2.88 | 0.028 |
| Average daily | 507 | 549 | 13.6 | 0.018 |
| Avg. daily feed | 1330 | 1270 | 45 | 0.21 |
| N | 12 | 12 | | |

### EXAMPLE 4

### 3-NPB Studies

It was hypothesized that boron mediates its effect in the pig by quadrivalent crosslinking. 3-nitrophenylboronic acid (3-NPB) which is an avid blocker of boron crosslinks was administered orally to pigs of about 100 kg body weight at the rates of 0 and 1 grams in combination with supplemental boron at the rates of 0 and 50 ppm in feed. The 3-NPB was administered for 10 days. The pigs were evaluated for lameness daily and euthanatized on day 13 and the joints and other organs were examined.

All of the pigs that received 3-NPB but no supplemental boron developed clinical manifestations of OCD within 10 days, but only one in 5 of the pigs receiving supplemental boron developed lameness when 3-NPB was provided (Table 3). A chi-square analysis indicated a significant (p<0.05) effect of 3-NPB in inducing lameness and a significant effect of supplemental boron in preventing the lameness induced by 3-NPB.

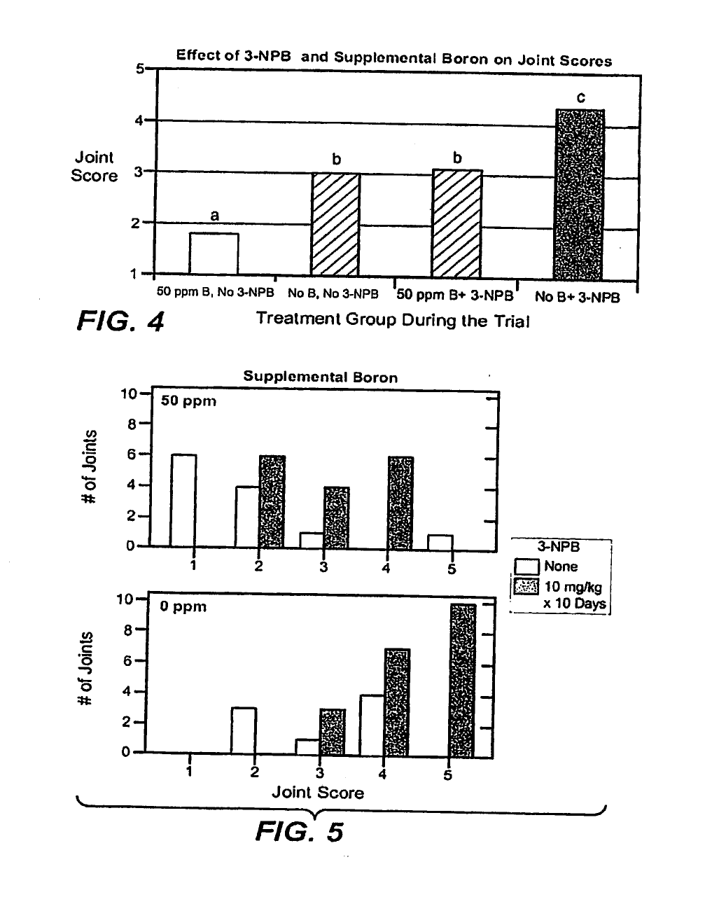

Examination of shoulder, stifle, hock and elbow joints showed that 3-NPB treated pigs with no supplemental boron had a higher prevalence of osteochondrosis lesions and a more intense severity of lesions than other treatment groups, with the lowest prevalence of lesions and the lowest severity among those pigs receiving supplemental boron and no 3NPB. Administration of 3-NPB along with boron resulted in a prevalence and severity of gross joint pathology similar to that observed in unsupplemented pigs. (FIGS. 4 and 5).

It was concluded that 3-NPB is acting as a competitive inhibitor of borate.

It is generally considered that the pig is the archetypic model species for osteochondrosis in mammals (see Reiland S. Osteochondrosis in the pig. Acta Radiol 1-118, 1975) The cascade of pathophysiologic events that culminate in clinical manifestations of osteochondrosis (OCD) in the pig are generally believed to be those events that occur in the other mammalian species that develop OCD, particularly the horse, dog, ruminants and humans. Since the pig is the model for OCD in other mammals and it has been demonstrated that boron is useful for prevention and treatment of OCD in the pig, it logically follows that boron should have a similar effect in other mammals and the effect should be mediated by a similar biochemical mechanism.

Therefore, it was concluded that administration of 3-NPB in species that are known to be susceptible to OCD but at a low prevalence, specifically cattle, horses, and dogs, should produce cartilage lesions indicating presence of a boron receptor moiety in cartilage.

Three healthy Holstein steer calves of average weight about 250 lbs and three healthy Quarter Horse fillies of average weight of about 500 lbs were administered 3-NPB at the rate of 10 mg/kg body weight per day. The 3-NPB was given to the calves by daily intraperitoneal injection, while the horses were administered the daily dose of 3-NPB mixed in feed. All animals consumed a standard ration of commercial feed and free-choice alfalfa-grass mixed hay.

Lameness was first observed in the calves at day 7 of the 3-NPB treatment. One calf was euthanatized at treatment day 14 and the other two at treatment day 21. Severe OCD lesions were visible in the hock and elbow joints of all calves with increasing severity noted with increasing time on 3-NPB.

Among the horses treated with oral 3-NPB, one filly showed clear signs of front leg lameness during exercise on treatment day 10 and was euthanatized on day 14. The other two horses were euthanatized on treatment day 28, at which time lameness was visible during exercise in one of the two horses. OCD lesions of varying degree were observed in the shoulder, elbow, fetlock and hock joints of all horses. Among the notable lesions were a 1 cm×1 cm necrotic lesion of the cartilage was found on the proximal articular surface of left front P1 at day 14, a developing flap lesion on the distal tibia at day 28 and in another horse, profound thinning of cartilage and obvious cartilage wear lines were observed in the left front fetlock and left elbow at day 28.

In experiments with normal healthy crossbred hounds of age 10 weeks (body weight 8 kg), the administration of 3-NPB as a single daily dose of 10 mg per kg body weight resulted in visible foreleg lameness as early as 12 days. Necropsy revealed gross lesions of necrosis and hemorrhage in the distal ulnar growth plate. There was also evidence of cartilage erosion in the articular surfaces of the distal tibia and the proximal ulna. The gross lesions in the distal ulnar growth plates resembled the pathologic changes associated with osteochondrosis in swine.

It is concluded that the pig is a suitable model for OCD in both ruminant and non-ruminant animals, including carnivores, and that boron receptor sites exist in all mammalian species, and that supplemental boron is expected to be an effective preventative and remedy in all mammalian species.

TABLE 3
3-NPB Clinical Lameness Score Level of Boron Supplementation
Crosstabulatlon Count

| Level of Boron | | | Clinical Lameness | | |
|---|---|---|---|---|---|
| [symbol] | | | Normal | Visible | Total |
| 0 | 3- | None | 6 | 0 | 6 |
| | | 1 gram daily × 10 | 0 | 4 | 4 |
| | Total | | 6 | 4 | 10 |
| 50 ppm | 3- | None | 5 | 0 | 5 |
| | | 1 gram daily × 10 | 4 | 1 | 5 |
| | Total | | 9 | 1 | 10 |

[symbol]

indicates data missing or illegible when filed

### EXAMPLE 5

### Use of Boron In Swine In Field Situations

Boron was added to the diet of sows at 1 mg/kg body weight as boric acid. There was no negative effect on reproduction or fertility. The sows appeared to have normal estrus activity and normal conception rate, with no negative effects on cyclic activity or on pigs born or on piglet viability. Three sows that were noticeably anl25ppmously lame became perfectly sound. Among a group of 95 piglets the birth to weaning mortality was 2 piglets. The usual mortality for this farm was about 5 to 7%. These piglets remained on boron 50 ppm plus ascorbate 125ppm. One pig was euthanized and examined at a weight of about 85 pounds. No abnormalities were observed in any of the joints. All bones of the appendicular skeleton were sectioned on the band saw. Bones had excellent mineralization and the growth plates were narrow and crisply demarcated, including the distal ulnar growth plate which is an early predilection site for OC-related abnormalities.

An Iowa farm treated a group of about 100 pigs with 50 ppm boron as boric acid. None of these pigs developed any signs of lameness or unsoundness. The farmer reported that these pigs are the most sound he has raised. The estimated previous lameness/unsoundness rate was about 25 to 30%, and zero in the test group. The pigs demonstrated excellent growth rate. The absence of lameness and hock swelling was observed. Two pigs with hock swelling were euthanised from among the younger and older pigs not treated with boric acid. The younger pig of about 50 pounds body weight showed evidence of early OC changes in the hock. An older pig of about 250 pounds body weight with severe lameness in the right hock was euthanised. Severe advanced OCD was observed in the hock, and growth plate abnormalities were observed when the bones were sectioned. Culture of the hock joints was negative, ruling out bacterial infection and indicating that OC is the likely cause of lameness.

1. A method of preventing or treating osteochondrosis comprising administering a therapeutically effective amount of a boron containing compound to a mammal in need of such treatment.

2. The method of claim 1 wherein the boron containing compound is administered prior to detection of osteochondrosis symptoms in said mammal.

3. The method of claim 1 wherein the mammal is a human.

4. The method of claim 1 wherein the mammal is selected from the group consisting of pigs, horses, mules, donkeys, cattle, sheep, goats, llamas, dogs, and cats.

5. The method of claim 1 wherein the therapeutically effective amount of the supplemental boron containing compound is from about 1 to about 500 ppm, from about 1 to about 150 ppm or about 50 ppm or from about 25 ppm to about 50 ppm.

6. The method of claim 1 wherein the supplemental boron containing compound is selected from organic boron containing compounds, inorganic boron containing compounds, boron containing minerals, plant material with elevated boron levels and microorganisms such as yeast with elevated boron levels.

7. The method of claim 6 wherein the inorganic boron containing compound is selected from the group consisting of sodium borate, boric acid, calcium borate, magnesium borate, halogen containing borate, ammonium borate, potassium borate, iron and magnesium containing borate, tantalum borate, beryllium borate, iron and nickel containing borate, carbonate containing borate, sodium and calcium containing borate, arsenate containing borate, calcium and rare earth containing borate, sulphate containing borate, magnesium and calcium containing borate, manganese borate, aluminum borate, calcium and strontium containing borate, phosphate containing borate, tin borate, strontium borate, zinc borate, calcium borosilicate, sodium borosilicate, aluminum borosilicate, calcium and rare earth containing borosilicate, lead borosilicate, barium borosilicate, lithium borosilicate, and sodium fluoroborat; wherein the organic boron containing compound is selected from the group consisting of complexes and compounds formed by boron with fructose, sorbitol, mannitol, xylitol, sorbose, threonine, methionine, modified starches, hydrolyzed starches, oxidized starches, non-modified starches, dextrins, amidated sugars, glucosamine, mannosamine, esters of glycerol fatty acids, salicylate complexes, salts of bisoxalato acid, calcium borosucrose, alcohols, alcohol amines, sugar acids, saccharic acid, gluconic acid, aminated sugar acids, and calcium borogluconate and wherein the boron containing mineral is selected from the group consisting of borax, colemanite, hydroboracite, kernite, ulexite, datolite, danburite, szaibelyite, suanite, inderite, sassolite, inyoite, probertite, howlite, ezcurrite, kurnakovite, meyerhofferite, priceite, nobleite, and searlesite.

8. The method of claim 1 wherein the therapeutically effective amount of a boron containing compound is administered by adding said therapeutically effective amount of boron containing compound to an animal feed composition.

9. The method of claim 1 wherein the therapeutically effective amount of a boron containing compound is administered by adding said therapeutically effective amount of boron containing compound to drinking water.

10. A method of increasing the efficiency of absorption of phosphorus in an animal or of decreasing the amount of phosphorus excreted by an animal, comprising administering supplemental boron to said animal.

11. The method of claim 10 wherein the supplemental boron is administered by adding said therapeutically effective amount of boron containing compound to an animal feed composition.

12. The method of claim 10 wherein the supplemental is administered by adding said therapeutically effective amount of boron containing compound to drinking water.

13. A boron containing supplement for addition to animal feed or drinking water to provide supplemental boron for the prevention and treatment of osteochondrosis in an animal.

14. The boron containing supplement of claim 13 wherein the addition of the boron containing supplement to the animal feed or drinking water increases the concentration of boron in said animal feed to about 25 ppm to about 500 ppm.

15. The use of a boron containing compound for the manufacture of a composition for preventing or treating osteochondrosis in a mammal.

16. The use of claim 15 wherein the composition is an animal feed composition comprising supplemental boron wherein the supplemental boron concentration in said animal feed is about 1 to about 500 ppm.

17. The use of claim 16 wherein the supplemental boron concentration is from about 1 to about 500 ppm, from about 1 to about 150 ppm or from about 25 ppm to about 50 ppm or about 50 ppm.
